(19) United States
(12) Patent Application Publication
(10) Pub. No.: US 2013/0065962 A1
(43) Pub. Date: Mar. 14, 2013

(54) Compounds For Use In The Treatment Of Diseases

(71) Applicants: Andreas Kubin, Wien (AT); Paul Furtmuller, Wien (AT); Gerhard Wolber, Wien (AT); Daniela Schuster, Wien (AT)

(72) Inventors: Andreas Kubin, Wien (AT); Paul Furtmuller, Wien (AT); Gerhard Wolber, Wien (AT); Daniela Schuster, Wien (AT)

(73) Assignee: INOXIA LIFESCIENCES GMBH, Wien (AT)

(21) Appl. No.: 13/578,516

(22) PCT Filed: Jan. 28, 2011
(86) PCT No.: PCT/AT11/00050

§ 371 Date: Aug. 10, 2012

(30) Foreign Application Priority Data
Jan. 29, 2010 (AT) A122/2010

(51) Int. Cl. A61K 31/165; A61P 11/06; A61P 25/00; A61P 43/00; A61P 1/04; C07C 229/40; A61P 11/02; A61P 17/00; A61P 31/22; A61P 9/00; A61P 9/12; C07C 243/34; A61K 31/198; A61P 1/00

(52) U.S. Cl. 514/567; 514/615; 564/149; 562/433; 564/150

(57) ABSTRACT

The present invention relates to a compound of the general formula (III):

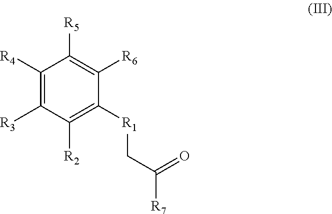

The present invention relates to compounds for treatment of inflammatory diseases related to eosinophil peroxidase.

Human enzymes of the class of peroxidases are part of the unspecific immune defense. They are released in high concentrations in the defense of pathogenic microorganisms and catalyze diverse oxidation reactions of bio-molecules, whereby intruders, like bacteria and viruses, are inactivated. In that, however, due to an overproduction of these proteins, there frequently also is oxidative damaging of the body's own tissues, and inflammations are the consequence.

Therefore, these enzymes are associated with many diseases, which play a significant role in our cultural area. These are so-called “auto-enzyme-induced” diseases, wherein in particular the body's own proteins MPO (myeloperoxidase) and EPO (eosinophil peroxidase; EC number: 1.11.1.7) are associated with the pathogenesis of many inflammatory diseases (see Table 1). In addition, milk contains lactoperoxidase (LPO), which has antimicrobial and antioxidant properties.

TABLE 1
Examples for “auto-enzyme-induced” diseases, in the course
of which peroxidases are involved by overproduction (also see Davies,
M J. et al. Antioxidants & Redox Signaling 10 (2008) 1199-1234).

| Disease | Enzyme |
|---|---|
| Asthma (chronic) | EPO |
| Smoker's lung (COPD) | MPO |
| Alzheimer's | MPO |
| Multiple sclerosis (MS) | MPO, EPO |
| Arteriosclerosis | MPO |
| Cystic fibrosis | EPO |
| Ulcerative colitis | EPO |
| Mastitis (vet. med.) | LPO |
| Cancer (following infections) | EPO |
| Hypertension (NO signal) | EPO |

Therefore, it is advantageous to develop specific inhibitors against MPO and EPO, the most prominent and most aggressive representatives of this class of enzymes, which inhibitors subsequently serve as the basis for new medication and therapies for inflammatory diseases.

EPO is considered the main cause for many diseases, in particular the chronic course of bronchial asthma. With a well tolerable inhibitor, for the first time, a real healing approach for chronic bronchial asthma could be provided. Something similar applies to multiple sclerosis, ulcerative colitis, cystic fibrosis and other inflammatory processes, in which EPO is involved as the main cause. These serious and in the western world highly increasing diseases mostly show a chronic course and so far could only be treated with very little success.

The body's own protein eosinophil peroxidase (EPO) is released, as soon as eosinophils (white blood cells, i.e. leukocytes) are stimulated (e.g. upon penetration of pathogenic substances or parasites, i.e. infections). Simultaneously, there is increased uptake of oxygen into the phagosome (“respiratory burst”) at the membrane-bound NADHP oxidase complex, whereby a number of reactive oxygen species (above all superoxide) are released. Subsequently, these are dismutated into hydrogen peroxide (H₂O₂) and reduced to water by eosinophil peroxidase (Mitra, S N. et al. Redox Report 5 (2000) 215-224).

With this EPO/H₂O₂ system, on the one hand, the physiological role of the enzyme takes effect (defense against pathogens), and on the other hand, it causes unspecific and specific cell damage.

Unspecific tissue damage includes the destruction of cells/cell walls, since EPO, due to the very high positive charge (pI>11), is able to penetrate the lipid membrane of cells. Therefore, on its way to the target locations of the infection, EPO destroys cells as well as tissues and thus causes inflammations.

Furthermore, eosinophils contribute to the pathogenesis of allergen-controlled diseases, like bronchial asthma. Bronchial asthma is an inflammation or increased sensitivity, respectively, of the mucous membranes of the bronchi, which results in narrowing of the airways. This clinical picture is based on the stimulation of certain defense cells, so-called mast cells, via cytokines, like interleukin 5 (IL 5). In case of asthma, mast cells and eosinophilic granulocytes are attracted in the bronchial area. These cells release substances (above all histamine), which, among other things, contract the muscles of the airways and stimulate the production of mucus in the lungs. This reaction mostly takes place very quickly, within 15 to 30 minutes after contact with the triggering substance and/or stress. Later (within two to four hours), inflammatory cells (eosinophilic granulocytes) then migrate into the walls of the bronchi and there cause the chronic form (inflammation). If these cells are stimulated, they release cytotoxic proteins, which promote many of the pathological characteristics of asthma: denaturation of the lung epithelium, destruction of the epithelium morphology, increased microvascular permeability and edemas. During the formation of chronic inflammation, however, molecules are likewise released, which are involved in the “remodeling” (regeneration) of tissue. Thereby, destroyed tissue is reproduced and the accumulation of “inelastic” connective tissue prevented.

Specific cell damage is caused by a number of aggressive oxidation products of EPO and diffusible free radicals, which are produced in the enzymatic reaction system EPO/H₂O₂. Due to the extraordinary redox potential of an enzyme intermediate (Compound I), EPO is able to oxidize diverse small molecules. These physiologically relevant enzyme substrates include nitrite (NO₂⁻), bromide (Br⁻) as well as the pseudohalide thiocyanate (SCN⁻). Subsequently, highly reactive substances are formed, like nitrogen dioxide radicals (NO₂.), hypobromite (⁻OBr) as well as hypothiocyanate (⁻OSCN) or cyanate (⁻OCN), respectively. Furthermore, it has to be pointed out that the biological consequences of the EPO/H₂O₂ system are highly substrate-specific. Thus, the physiological serum concentration of SCN⁻ is substantially higher (or can be favorably influenced nutritionally, respectively) than that of Br⁻ or NO₂⁻. Thus, for example, the oxidation product ⁻OSCN activates the transcription factor NF-κB substantially stronger than NO₂. and therefore has a more pro-inflammatory effect in the MAP kinase system (Wang, J. et al. Arch Biochem Biophys 445 (2006) 256-260). Now, these highly active reaction products, on the one hand, act as part of the passive immune defense and attack large parasites penetrated into the body, whereby they fulfill the physiological role of EPO.

On the other hand, these substances can attack large bio-molecules (e.g. lipids, proteins, DNA, RNA) in non-enzymatic reactions, whereby these are modified in their structure and/or functionality. Bromine or nitro groups are integrated, especially at hydroxy and amino groups (bromo- and nitrotyrosines, bromohydrines, bromoaldehydes, bromonucleotides, lipid peroxides). Thus, for example, in the sputum of asthma patients, 3-bromotyrosines (biomarkers) could be detected (Aldridge, C J. et al. Free Radical Biology & Medicine 33 (2002) 6, 847-856).

In other cases, a significant conformity of chronic infections/inflammations and the pathogenesis of cancer could be detected, which can be ascribed to oxidative damage at the DNA (e.g. Schistosoma haematobium and cancer of the bladder, or Opisthorcis vicerrini and cholangiocarcinoma (cancer of the bile duct) (Mitra, S N. et al. Redox Report 5 (2000) 215-224).

Furthermore, EPO is involved in the biochemistry of the vasoactive, i.e. vasodilating, substance nitrogen monoxide (NO), which plays a substantial role in angiogenesis, regulation of the blood pressure, dilation of the bronchi (e.g. in newborns) as well as other physiological phenomena. It is assumed that NO oxidized by EPO Compound I and Compound II is released as NO and reacts with superoxide to peroxynitrite (ONOO⁻). In turn, this highly reactive compound (a marker for oxidative stress) attacks lipids and proteins, whereby nitrotyrosines and lipid peroxides are formed. On the other hand, by capturing NO, this important regulatory diatomic signal molecule is no longer available, whereby important biological functions (e.g. as transmitter) can no longer be fulfilled or only partially fulfilled (Abu-Soud, H M. et al. Biochem 40 (2001) 11866-11875).

The occurrence of such symptoms verifies that the plasma or tissue concentration, respectively, of eosinophil peroxidase or its “fingerprint”, respectively, at reaction products (e.g. brominated lipids and proteins) correlates with the degree of the disease. Eosinophils as well as eosinophil peroxidase can be found in blood, sputum, bronchial tissue and the bronchoalveolar lavage of asthmatics, and today serve medicine as a direct, quantifiable marker of asthma as well as indirect indicator of an inflammation and the response of a patient to asthma therapies.

WO 2008/121670 describes pyrimidinylhydrazides and their use in the treatment of bronchial asthma.

WO 00/073280 describes catechin-substituted hydrazones and their use in the treatment of bronchial asthma.

WO 2009/145360 relates to phenyl or thiophene derivatives, respectively, which likewise can be used for the treatment of bronchial asthma.

WO 2004/080377 discloses phenylhydrazides, which are suited to modulate potassium channels in cells, whereby, among other things, diseases like bronchial asthma can be treated.

US 2003/0225102 and WO 2002/006224 describe hydrazides substituted with a heterocyclic substituent. These compounds can be used for the treatment of bronchial asthma.

WO 2007/026215, WO 2005/123688, DE 10 2006 005 179, U.S. Pat. No. 5,571,846, EP 0 323 590, WO 01/032156, WO 2005/085185 and U.S. Pat. No. 4,082,846 describe compounds with a hydrazine structure, which are suited for use in the treatment of most different diseases.

It is one object of the present invention to provide compounds, which are able to significantly or entirely inhibit the activity of eosinophil peroxidase.

Surprisingly, it was found that certain compounds like hydrazides are able to inhibit the activity of eosinophil peroxidase. Therefore, the present invention relates to compounds of the general formula (III):

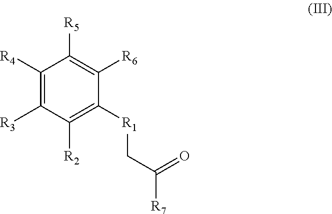

for use in the treatment and/or prevention of diseases, in particular inflammatory diseases, which are related to eosinophil peroxidase, wherein

R₁ is CH₂, NH, O, S or a single bond,

R₂, R₃, R₄, R₅ and R₆ independently of one another are H, OH, F, Cl, Br, I or a C₁ to C₅ alkyl group, and

R₇ is H, OH, NH₂, NH—NH₂ or CH₃.

A further aspect of the present invention relates to hydrazides of the general formula (I):

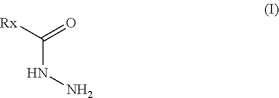

for treatment and/or prevention of diseases, in particular inflammatory diseases, which are related to eosinophil peroxidase, wherein, according to the invention, Rx is a heterocyclic compound (heterocyclic residue), like pyridine, indole, pyrazole or pyrimidine, or an aromatic compound (aromatic residue), like naphthol, benzene or phenylaminoethane.

For the inhibitory activity of the compounds according to the invention, the free terminal amino group is advantageous, which acts as electron acceptor.

Furthermore, however, steric and/or electrochemical properties of this compound are also responsible for the binding and/or enzymatic reaction of these compounds with EPO. A pharmacophoric model showed that the substances according to the invention must have various motifs (e.g. hydrogen bond donors, hydrogen bond acceptors, aromatic rings/areas, hydrophobic areas). Therefrom results the following exemplary structure, which also considers bond lengths and domains (II):

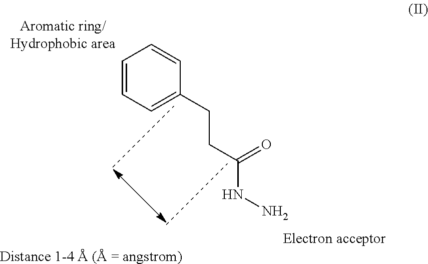 [SMILES: NNC(=O)CCc1ccccc1]

The compounds according to the invention, in particular the phenylaminoethane hydrazides (PAEHs), which are particularly preferred, and their derivatives correspond to this model, wherein in this case the distance between benzene ring and acid hydrazide group is 2.65 Å (IIIa):

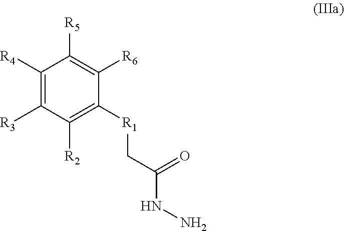

Substituent R₁ is CH₂, NH, O, S or a single bond, and the substituents R₂, R₃, R₄, R₅ and R₆ are independently of one another H, F, Cl, Br, I or a C₁ to C₅ alkyl group, R₇ is H, OH, NH₂, NH—NH₂ or CH₃.

A central key role in the production of the aggressive, cell-damaging substances plays—as initially discussed already—eosinophil peroxidase, EPO. These processes, in particular inflammatory processes, in which EPO is involved, can be inhibited by using the substances according to the invention, so that diseases, which are related to eosinophil peroxidase, can be treated.

The compounds according to the invention are selective for eosinophil peroxidase (presence in white blood cells) and homologous lactoperoxidase (presence in breast milk and in saliva). These compounds, however, are not able to inhibit myeloperoxidase, in particular human myeloperoxidase, to the same extent, which enables the targeted use of these compounds as specific medication, selectively against EPO.

Due to the strong inhibitory effect of the substances according to the invention, it is in fact possible to develop therapeutic applications with very low dosages. In that, local or systemic concentrations of about 0.001 to 10 μM can be sufficient.

The compounds according to the invention are sufficiently known to the skilled person and are manufactured according to known methods (see, e.g., Finger, G C. et al. J Am Chem Soc 81 (1959) 94-101). Most N-arylglycines are just like their esters, hydrazides and other derivatives manufactured for biological examination of their tuberculostatic potential. p-alkylanilines and p-cyclohexylanilines are manufactured by means of Beckmann rearrangement of oximes of the corresponding p-substituted acetophenones. p-alkoxyanilines are manufactured by means of alkylation of p-benzalaminophenol with alkyl halides and NaOH in aqueous ethanol with subsequent hydrolysis of the aldimines with HCl (Tien, N B. et al. Org Chem 23 (1958) 186-8).

The term “diseases, in particular inflammatory diseases, which are related to eosinophil peroxidase” refers to diseases and conditions, which can be attributed to an increased activity of EPO in an individual (see, e.g., Davies, M J. et al. Antioxidants & Redox Signaling 10 (2008) 1199-1234; Wang, J. et al. Arch Biochem Biophys 445 (2006) 256-260; Mitra, S N. et al. Redox Report 5 (2000) 215-224). Such diseases are by all means known to the skilled person, as this was also discussed initially. The connection between the EPO activity and diseases, which are a consequence of the EPO activity, is likewise sufficiently known to the skilled person. For example, in the sputum of patients suffering from bronchial asthma, 3-bromotyrosines (biomarkers) could be detected using GC-MS (gas chromatography mass spectroscopy), which were formed by modification of proteins by means of ⁻OBr, an EPO oxidation product (Aldridge, C J. et al. Free Radical Biology & Medicine 33 (2002) 847-856).

Hypothiocyanate (⁻OSCN) or NO₂., respectively, reaction products of EPO, activate the transcription factor NF-κB and therefore have a pro-inflammatory effect in the MAP kinase system. Transgenic mice (EPO knock-out) showed substantially lower damaging by ulcerative colitis. This also applies to other chronic inflammations like Crohn's disease or cystic fibrosis (Wang, J. et al. Arch Biochem Biophys 445 (2006) 256-260).

Tumor diseases, too, can be a consequence of increased EPO activity, since this results in oxidative damaging of the DNA, which is caused by reactive oxygen species (e.g. bromonucleotides, singlet oxygen) following infections (e.g. Schistosoma haematobium and cancer of the bladder, or Opisthorcis vicerrini and cholangiocarcinoma (cancer of the bile duct) (Mitra et al. Redox Report 5 (2000) 215-224). An alternative designation for “diseases, in particular inflammatory diseases, which are related to eosinophil peroxidase” are diseases based on an increased activity of EPO in the body, wherein the increased activity refers to an average individual not suffering from any diseases representing a consequence of increased EPO activity.

By migration of EPO or its reactive oxidation products (⁻OBr or NO₂., respectively), respectively, lipid double layers as well as membrane proteins and cell walls are modified (bromo- and nitrotyrosines, lipid peroxides), disintegrated and ultimately destroyed (Wang, J. et al. Arch Biochem Biophys 445 (2006) 256-260). Thus results in tissue damaging and necroses. Using the selective inhibitors, the tissue-damaging effect of EPO is prevented and simultaneously, however, the tissue-forming function of the eosinophilic granulocytes maintained. Thus, e.g., the so far irreversible and chronic course of bronchial asthma (EPO inhibitor) can be stopped, and even a healing approach can be given with this new drug group.

The compounds according to the invention comprise, among others, pharmaceutically acceptable acid addition salts, by which according to the invention such salts must be understood, which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, wherein the salts of hydrochloric acid, hydrobromic acid, sulphuric acid, phosphoric acid and acetic acid are particularly preferred.

It was found out that it is advantageous, if R₇ has a free amino group, preferably a hydrazide group. The amino groups of such compounds of the general formulas (I) or (IIIa), respectively, and (IV) are advantageous for their effect as EPO inhibitor. I.e., the compounds according to the invention should have the free amino group at the site of action. It is, however, possible, in order to increase tolerability of the compounds according to the invention, to provide the amino group with a protective group, which is removed at the site of action, if necessary (prodrug concept). Of course, R₇ of the compounds of the general formula (III) may also be H, OH or CH₃ residues. Such compounds, too, are able to inhibit eosinophil peroxidase with high effectiveness.

According to a particularly preferred embodiment of the present invention, R₁ is NH, wherein the hydrazide has the general formula (IV):

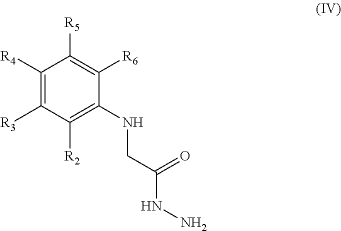

According to a preferred embodiment of the present invention, the C₁ to C₅ alkyl group is selected from the group consisting of CH₃ and CH₂CH₃.

According to a further preferred embodiment of the present invention, R₁ is CH₂, NH, O or S, particularly preferred NH or O, R₂ is F or H, R₃ is Cl, Br or H, R₄ is Cl, F, CH₃ or H, R₅ and R₆ are H, and R₇ is OH or NH—NH₂.

According to a particularly preferred embodiment of the present invention, the compound (III) according to the invention has the following substituents (see Table A):

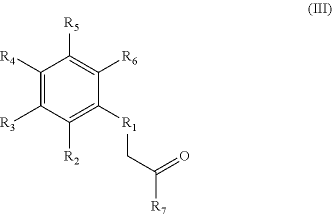

TABLE A

| No. | R₁ | R₂ | R₃ | R₄ | R₅ | R₆ | R₇ |
|---|---|---|---|---|---|---|---|
| 1 | NH | H | H | F | H | H | NH—NH₂ |
| 2 | NH | H | H | Cl | H | H | NH—NH₂ |
| 3 | NH | F | H | H | H | H | NH—NH₂ |
| 4 | NH | H | Cl | F | H | H | NH—NH₂ |
| 5 | NH | H | Br | H | H | H | NH—NH₂ |
| 6 | NH | H | H | H | H | H | NH—NH₂ |
| 7 | NH | F | H | F | H | H | NH—NH₂ |
| 8 | NH | H | H | CH₃ | H | H | NH—NH₂ |
| 9 | NH | F | Cl | H | H | H | NH—NH₂ |
| 10 | NH | Cl | H | H | H | H | NH—NH₂ |
| 11 | NH | F | H | H | Cl | H | NH—NH₂ |
| 12 | NH | F | H | Cl | H | H | NH—NH₂ |
| 13 | NH | F | H | H | H | Cl | NH—NH₂ |
| 14 | NH | H | Cl | H | H | H | NH—NH₂ |
| 15 | NH | H | F | H | H | H | NH—NH₂ |
| 16 | NH | F | F | H | H | H | NH—NH₂ |
| 17 | NH | H | F | F | H | H | NH—NH₂ |
| 18 | NH | H | F | H | F | H | NH—NH₂ |
| 19 | NH | H | F | H | H | F | NH—NH₂ |
| 20 | NH | CH₃ | H | H | H | H | NH—NH₂ |
| 21 | NH | H | CH₃ | H | H | H | NH—NH₂ |
| 22 | NH | H | H | H | CH₃ | H | NH—NH₂ |
| 23 | NH | H | H | H | H | CH₃ | NH—NH₂ |
| 24 | NH | CH₂CH₃ | H | H | H | H | NH—NH₂ |
| 25 | NH | H | CH₂CH₃ | H | H | H | NH—NH₂ |
| 26 | NH | H | H | CH₂CH₃ | H | H | NH—NH₂ |
| 27 | NH | H | H | H | CH₂CH₃ | H | NH—NH₂ |
| 28 | NH | H | H | H | H | CH₂CH₃ | NH—NH₂ |
| 29 | NH | F | H | CH₃ | H | H | NH—NH₂ |
| 30 | NH | F | CH₃ | H | H | H | NH—NH₂ |
| 31 | NH | F | Br | H | H | H | NH—NH₂ |
| 32 | NH | F | H | Br | H | H | NH—NH₂ |
| 33 | NH | F | H | H | Br | H | NH—NH₂ |
| 34 | NH | F | H | H | H | Br | NH—NH₂ |
| 35 | NH | H | F | H | Br | H | NH—NH₂ |
| 36 | NH | H | F | Br | H | H | NH—NH₂ |
| 37 | NH | Br | F | H | H | H | NH—NH₂ |
| 38 | NH | Br | H | Cl | H | H | NH—NH₂ |
| 39 | NH | Br | H | H | Cl | H | NH—NH₂ |
| 40 | NH | Br | Cl | H | H | H | NH—NH₂ |
| 41 | NH | Br | H | H | H | Cl | NH—NH₂ |
| 42 | NH | H | Br | F | H | H | NH—NH₂ |
| 43 | NH | H | Br | H | F | H | NH—NH₂ |
| 44 | NH | H | Br | Cl | H | H | NH—NH₂ |
| 45 | NH | H | Br | H | Cl | H | NH—NH₂ |
| 46 | NH | H | Br | H | H | Cl | NH—NH₂ |
| 47 | NH | Cl | H | H | F | H | NH—NH₂ |
| 48 | NH | Cl | F | H | H | H | NH—NH₂ |
| 49 | NH | Cl | H | F | H | H | NH—NH₂ |
| 50 | NH | Cl | H | H | F | H | NH—NH₂ |
| 51 | NH | Cl | H | H | H | F | NH—NH₂ |
| 52 | NH | H | Cl | H | F | H | NH—NH₂ |
| 53 | NH | H | Cl | H | F | H | NH—NH₂ |
| 54 | NH | H | Cl | H | H | F | NH—NH₂ |
| 55 | NH | H | H | Cl | F | H | NH—NH₂ |
| 56 | NH | H | H | Cl | H | F | NH—NH₂ |
| 57 | NH | H | H | H | H | F | NH—NH₂ |
| 58 | O | H | H | F | H | H | NH—NH₂ |
| 59 | O | H | H | Cl | H | H | NH—NH₂ |
| 60 | O | F | H | H | H | H | NH—NH₂ |
| 61 | O | H | Cl | F | H | H | NH—NH₂ |
| 62 | O | H | Br | H | H | H | NH—NH₂ |
| 63 | O | H | H | H | H | H | NH—NH₂ |
| 64 | O | F | H | F | H | H | NH—NH₂ |
| 65 | O | H | H | CH₃ | H | H | NH—NH₂ |
| 66 | O | F | Cl | H | H | H | NH—NH₂ |
| 67 | O | Cl | H | H | H | H | NH—NH₂ |
| 68 | O | F | H | H | Cl | H | NH—NH₂ |
| 69 | O | F | H | Cl | H | H | NH—NH₂ |
| 70 | O | F | H | H | H | Cl | NH—NH₂ |
| 71 | O | H | F | H | H | H | NH—NH₂ |
| 72 | O | F | F | H | H | H | NH—NH₂ |
| 73 | O | H | F | F | H | H | NH—NH₂ |
| 74 | O | H | F | H | F | H | NH—NH₂ |
| 75 | O | H | F | H | H | F | NH—NH₂ |
| 76 | O | CH₃ | H | H | H | H | NH—NH₂ |
| 77 | O | H | CH₃ | H | H | H | NH—NH₂ |
| 78 | O | H | H | H | CH₃ | H | NH—NH₂ |
| 79 | O | H | H | H | H | CH₃ | NH—NH₂ |
| 80 | O | CH₂CH₃ | H | H | H | H | NH—NH₂ |
| 81 | O | H | CH₂CH₃ | H | H | H | NH—NH₂ |
| 82 | O | H | H | CH₂CH₃ | H | H | NH—NH₂ |
| 83 | O | H | H | H | CH₂CH₃ | H | NH—NH₂ |
| 84 | O | H | H | H | H | CH₂CH₃ | NH—NH₂ |
| 85 | O | F | H | CH₃ | H | H | NH—NH₂ |
| 86 | O | F | CH₃ | H | H | H | NH—NH₂ |
| 87 | O | F | Br | H | H | H | NH—NH₂ |
| 88 | O | F | H | Br | H | H | NH—NH₂ |
| 89 | O | F | H | H | Br | H | NH—NH₂ |
| 90 | O | F | H | H | H | Br | NH—NH₂ |
| 91 | O | H | F | H | Br | H | NH—NH₂ |
| 92 | O | H | F | Br | H | H | NH—NH₂ |
| 93 | O | Br | F | H | H | H | NH—NH₂ |
| 94 | O | Br | H | Cl | H | H | NH—NH₂ |
| 95 | O | Br | H | H | Cl | H | NH—NH₂ |
| 96 | O | Br | Cl | H | H | H | NH—NH₂ |
| 97 | O | Br | H | H | H | Cl | NH—NH₂ |
| 98 | O | H | Br | F | H | H | NH—NH₂ |
| 99 | O | H | Br | H | F | H | NH—NH₂ |
| 100 | O | H | Br | Cl | H | H | NH—NH₂ |
| 101 | O | H | Br | H | Cl | H | NH—NH₂ |
| 102 | O | H | Br | H | H | Cl | NH—NH₂ |
| 103 | O | Cl | H | H | F | H | NH—NH₂ |
| 104 | O | Cl | F | H | H | H | NH—NH₂ |
| 105 | O | Cl | H | F | H | H | NH—NH₂ |
| 106 | O | Cl | H | H | F | H | NH—NH₂ |
| 107 | O | Cl | H | H | H | F | NH—NH₂ |
| 108 | O | H | Cl | F | H | H | NH—NH₂ |
| 109 | O | H | Cl | H | F | H | NH—NH₂ |
| 110 | O | H | Cl | H | H | F | NH—NH₂ |
| 111 | O | H | H | Cl | F | H | NH—NH₂ |
| 112 | O | H | H | Cl | H | F | NH—NH₂ |
| 113 | CH₂ | H | H | F | H | H | NH—NH₂ |
| 114 | CH₂ | H | H | Cl | H | H | NH—NH₂ |
| 115 | CH₂ | F | H | H | H | H | NH—NH₂ |
| 116 | CH₂ | H | Cl | F | H | H | NH—NH₂ |
| 117 | CH₂ | H | Br | H | H | H | NH—NH₂ |
| 118 | CH₂ | H | H | H | H | H | NH—NH₂ |
| 119 | CH₂ | F | H | F | H | H | NH—NH₂ |
| 120 | CH₂ | H | H | CH₃ | H | H | NH—NH₂ |
| 121 | CH₂ | F | Cl | H | H | H | NH—NH₂ |
| 122 | CH₂ | Cl | H | H | H | H | NH—NH₂ |
| 123 | CH₂ | F | H | H | Cl | H | NH—NH₂ |
| 124 | CH₂ | F | H | Cl | H | H | NH—NH₂ |
| 125 | CH₂ | F | H | H | H | Cl | NH—NH₂ |
| 126 | CH₂ | H | F | H | H | H | NH—NH₂ |
| 127 | CH₂ | F | F | H | H | H | NH—NH₂ |
| 128 | CH₂ | H | F | F | H | H | NH—NH₂ |
| 129 | CH₂ | H | F | H | F | H | NH—NH₂ |
| 130 | CH₂ | H | F | H | H | F | NH—NH₂ |
| 131 | CH₂ | CH₃ | H | H | H | H | NH—NH₂ |
| 132 | CH₂ | H | CH₃ | H | H | H | NH—NH₂ |
| 133 | CH₂ | H | H | H | CH₃ | H | NH—NH₂ |
| 134 | CH₂ | H | H | H | H | CH₃ | NH—NH₂ |
| 135 | CH₂ | CH₂CH₃ | H | H | H | H | NH—NH₂ |
| 136 | CH₂ | H | CH₂CH₃ | H | H | H | NH—NH₂ |
| 137 | CH₂ | H | H | CH₂CH₃ | H | H | NH—NH₂ |
| 138 | CH₂ | H | H | H | CH₂CH₃ | H | NH—NH₂ |
| 139 | CH₂ | H | H | H | H | CH₂CH₃ | NH—NH₂ |
| 140 | CH₂ | F | H | CH₃ | H | H | NH—NH₂ |
| 141 | CH₂ | F | CH₃ | H | H | H | NH—NH₂ |
| 142 | CH₂ | F | Br | H | H | H | NH—NH₂ |
| 143 | CH₂ | F | H | Br | H | H | NH—NH₂ |
| 144 | CH₂ | F | H | H | Br | H | NH—NH₂ |
| 145 | CH₂ | F | H | H | H | Br | NH—NH₂ |
| 146 | CH₂ | H | F | H | Br | H | NH—NH₂ |
| 147 | CH₂ | H | F | Br | H | H | NH—NH₂ |
| 148 | CH₂ | Br | F | H | H | H | NH—NH₂ |
| 149 | CH₂ | Br | H | Cl | H | H | NH—NH₂ |
| 150 | CH₂ | Br | H | H | Cl | H | NH—NH₂ |
| 151 | CH₂ | Br | Cl | H | H | H | NH—NH₂ |
| 152 | CH₂ | Br | H | H | H | Cl | NH—NH₂ |
| 153 | CH₂ | H | Br | F | H | H | NH—NH₂ |
| 154 | CH₂ | H | Br | H | F | H | NH—NH₂ |
| 155 | CH₂ | H | Br | Cl | H | H | NH—NH₂ |
| 156 | CH₂ | H | Br | H | Cl | H | NH—NH₂ |
| 157 | CH₂ | H | Br | H | H | Cl | NH—NH₂ |
| 158 | CH₂ | Cl | H | H | F | H | NH—NH₂ |
| 159 | CH₂ | Cl | F | H | H | H | NH—NH₂ |
| 160 | CH₂ | Cl | H | F | H | H | NH—NH₂ |
| 161 | CH₂ | Cl | H | H | F | H | NH—NH₂ |
| 162 | CH₂ | Cl | H | H | H | F | NH—NH₂ |
| 163 | CH₂ | H | Cl | F | H | H | NH—NH₂ |
| 164 | CH₂ | H | Cl | H | F | H | NH—NH₂ |
| 165 | CH₂ | H | Cl | H | H | F | NH—NH₂ |
| 166 | CH₂ | H | H | Cl | F | H | NH—NH₂ |
| 167 | CH₂ | H | H | Cl | H | F | NH—NH₂ |
| 168 | CH₂ | H | H | H | H | OH | NH—NH₂ |
| 169 | NH | H | H | F | H | H | OH |
| 170 | NH | H | H | Cl | H | H | OH |
| 171 | NH | F | H | H | H | H | OH |
| 172 | NH | H | Cl | F | H | H | OH |
| 173 | NH | H | Br | H | H | H | OH |
| 174 | NH | H | H | H | H | H | OH |
| 175 | NH | F | H | F | H | H | OH |
| 176 | NH | H | H | CH₃ | H | H | OH |
| 177 | NH | F | Cl | H | H | H | OH |
| 178 | NH | Cl | H | H | H | H | OH |
| 179 | NH | F | H | H | Cl | H | OH |
| 180 | NH | F | H | Cl | H | H | OH |
| 181 | NH | F | H | H | H | Cl | OH |
| 182 | NH | H | Cl | H | H | H | OH |
| 183 | NH | H | F | H | H | H | OH |
| 184 | NH | F | F | H | H | H | OH |
| 185 | NH | H | F | F | H | H | OH |
| 186 | NH | H | F | H | F | H | OH |
| 187 | NH | H | F | H | H | F | OH |
| 188 | NH | CH₃ | H | H | H | H | OH |
| 189 | NH | H | CH₃ | H | H | H | OH |
| 190 | NH | H | H | H | CH₃ | H | OH |
| 191 | NH | H | H | H | H | CH₃ | OH |
| 192 | NH | CH₂CH₃ | H | H | H | H | OH |
| 193 | NH | H | CH₂CH₃ | H | H | H | OH |
| 194 | NH | H | H | CH₂CH₃ | H | H | OH |
| 195 | NH | H | H | H | CH₂CH₃ | H | OH |
| 196 | NH | H | H | H | H | CH₂CH₃ | OH |
| 197 | NH | F | H | CH₃ | H | H | OH |
| 198 | NH | F | CH₃ | H | H | H | OH |
| 199 | NH | F | Br | H | H | H | OH |
| 200 | NH | F | H | Br | H | H | OH |
| 201 | NH | F | H | H | Br | H | OH |
| 202 | NH | F | H | H | H | Br | OH |
| 203 | NH | H | F | H | Br | H | OH |
| 204 | NH | H | F | Br | H | H | OH |
| 205 | NH | Br | F | H | H | H | OH |
| 206 | NH | Br | H | Cl | H | H | OH |
| 207 | NH | Br | H | H | Cl | H | OH |
| 208 | NH | Br | Cl | H | H | H | OH |
| 209 | NH | Br | H | H | H | Cl | OH |
| 210 | NH | H | Br | F | H | H | OH |
| 211 | NH | H | Br | H | F | H | OH |
| 212 | NH | H | Br | Cl | H | H | OH |
| 213 | NH | H | Br | H | Cl | H | OH |
| 214 | NH | H | Br | H | H | Cl | OH |
| 215 | NH | Cl | H | H | F | H | OH |
| 216 | NH | Cl | F | H | H | H | OH |
| 217 | NH | Cl | H | F | H | H | OH |
| 218 | NH | Cl | H | H | F | H | OH |
| 219 | NH | Cl | H | H | H | F | OH |
| 220 | NH | H | Cl | F | H | H | OH |
| 221 | NH | H | Cl | H | F | H | OH |
| 222 | NH | H | Cl | H | H | F | OH |
| 223 | NH | H | H | Cl | F | H | OH |
| 224 | NH | H | H | Cl | H | F | OH |
| 225 | NH | H | H | H | H | F | OH |
| 226 | O | H | H | F | H | H | OH |
| 227 | O | H | H | Cl | H | H | OH |
| 228 | O | F | H | H | H | H | OH |
| 229 | O | H | Cl | F | H | H | OH |
| 230 | O | H | Br | H | H | H | OH |
| 231 | O | H | H | H | H | H | OH |
| 232 | O | F | H | F | H | H | OH |
| 233 | O | H | H | CH₃ | H | H | OH |
| 234 | O | F | Cl | H | H | H | OH |
| 235 | O | Cl | H | H | H | H | OH |
| 236 | O | F | H | H | Cl | H | OH |
| 237 | O | F | H | Cl | H | H | OH |
| 238 | O | F | H | H | H | Cl | OH |
| 239 | O | H | F | H | H | H | OH |
| 240 | O | F | F | H | H | H | OH |
| 241 | O | H | F | F | H | H | OH |
| 242 | O | H | F | H | F | H | OH |
| 243 | O | H | F | H | H | F | OH |
| 244 | O | CH₃ | H | H | H | H | OH |
| 245 | O | H | CH₃ | H | H | H | OH |
| 246 | O | H | H | H | CH₃ | H | OH |
| 247 | O | H | H | H | H | CH₃ | OH |
| 248 | O | CH₂CH₃ | H | H | H | H | OH |
| 249 | O | H | CH₂CH₃ | H | H | H | OH |
| 250 | O | H | H | CH₂CH₃ | H | H | OH |
| 251 | O | H | H | H | CH₂CH₃ | H | OH |
| 252 | O | H | H | H | H | CH₂CH₃ | OH |
| 253 | O | F | H | CH₃ | H | H | OH |
| 254 | O | F | CH₃ | H | H | H | OH |
| 255 | O | F | Br | H | H | H | OH |
| 256 | O | F | H | Br | H | H | OH |
| 257 | O | F | H | H | Br | H | OH |
| 258 | O | F | H | H | H | Br | OH |
| 259 | O | H | F | H | Br | H | OH |
| 260 | O | H | F | Br | H | H | OH |
| 261 | O | Br | F | H | H | H | OH |
| 262 | O | Br | H | Cl | H | H | OH |
| 263 | O | Br | H | H | Cl | H | OH |
| 264 | O | Br | Cl | H | H | H | OH |
| 265 | O | Br | H | H | H | Cl | OH |
| 266 | O | H | Br | F | H | H | OH |
| 267 | O | H | Br | H | F | H | OH |
| 268 | O | H | Br | Cl | H | H | OH |
| 269 | O | H | Br | H | Cl | H | OH |
| 270 | O | H | Br | H | H | Cl | OH |
| 271 | O | Cl | H | H | F | H | OH |
| 272 | O | Cl | F | H | H | H | OH |
| 273 | O | Cl | H | F | H | H | OH |
| 274 | O | Cl | H | H | F | H | OH |
| 275 | O | Cl | H | H | H | F | OH |
| 276 | O | H | Cl | F | H | H | OH |
| 277 | O | H | Cl | H | F | H | OH |
| 278 | O | H | Cl | H | H | F | OH |
| 279 | O | H | H | Cl | F | H | OH |
| 280 | O | H | H | Cl | H | F | OH |
| 281 | CH₂ | H | H | F | H | H | OH |
| 282 | CH₂ | H | H | Cl | H | H | OH |
| 283 | CH₂ | F | H | H | H | H | OH |
| 284 | CH₂ | H | Cl | F | H | H | OH |
| 285 | CH₂ | H | Br | H | H | H | OH |
| 286 | CH₂ | H | H | H | H | H | OH |
| 287 | CH₂ | F | H | F | H | H | OH |
| 288 | CH₂ | H | H | CH₃ | H | H | OH |
| 289 | CH₂ | F | Cl | H | H | H | OH |
| 290 | CH₂ | Cl | H | H | H | H | OH |
| 291 | CH₂ | F | H | H | Cl | H | OH |
| 292 | CH₂ | F | H | Cl | H | H | OH |
| 293 | CH₂ | F | H | H | H | Cl | OH |
| 294 | CH₂ | H | F | H | H | H | OH |
| 295 | CH₂ | F | F | H | H | H | OH |
| 296 | CH₂ | H | F | F | H | H | OH |
| 297 | CH₂ | H | F | H | F | H | OH |
| 298 | CH₂ | H | F | H | H | F | OH |
| 299 | CH₂ | CH₃ | H | H | H | H | OH |
| 300 | CH₂ | H | CH₃ | H | H | H | OH |
| 301 | CH₂ | H | H | H | CH₃ | H | OH |
| 302 | CH₂ | H | H | H | H | CH₃ | OH |
| 303 | CH₂ | CH₂CH₃ | H | H | H | H | OH |
| 304 | CH₂ | H | CH₂CH₃ | H | H | H | OH |
| 305 | CH₂ | H | H | CH₂CH₃ | H | H | OH |
| 306 | CH₂ | H | H | H | CH₂CH₃ | H | OH |
| 307 | CH₂ | H | H | H | H | CH₂CH₃ | OH |
| 308 | CH₂ | F | H | CH₃ | H | H | OH |
| 309 | CH₂ | F | CH₃ | H | H | H | OH |
| 310 | CH₂ | F | Br | H | H | H | OH |
| 311 | CH₂ | F | H | Br | H | H | OH |
| 312 | CH₂ | F | H | H | Br | H | OH |
| 313 | CH₂ | F | H | H | H | Br | OH |
| 314 | CH₂ | H | F | H | Br | H | OH |
| 315 | CH₂ | H | F | Br | H | H | OH |
| 316 | CH₂ | Br | F | H | H | H | OH |
| 317 | CH₂ | Br | H | Cl | H | H | OH |
| 318 | CH₂ | Br | H | H | Cl | H | OH |
| 319 | CH₂ | Br | Cl | H | H | H | OH |
| 320 | CH₂ | Br | H | H | H | Cl | OH |
| 321 | CH₂ | H | Br | F | H | H | OH |
| 322 | CH₂ | H | Br | H | F | H | OH |
| 323 | CH₂ | H | Br | Cl | H | H | OH |
| 324 | CH₂ | H | Br | H | Cl | H | OH |
| 325 | CH₂ | H | Br | H | H | Cl | OH |
| 326 | CH₂ | Cl | H | H | F | H | OH |
| 327 | CH₂ | Cl | F | H | H | H | OH |
| 328 | CH₂ | Cl | H | F | H | H | OH |
| 329 | CH₂ | Cl | H | H | F | H | OH |
| 330 | CH₂ | Cl | H | H | H | F | OH |
| 331 | CH₂ | H | Cl | F | H | H | OH |
| 332 | CH₂ | H | Cl | H | F | H | OH |
| 333 | CH₂ | H | Cl | H | H | F | OH |
| 334 | CH₂ | H | H | Cl | F | H | OH |
| 335 | CH₂ | H | H | Cl | H | F | OH |
| 336 | CH₂ | H | H | H | H | OH | OH |

According to a preferred embodiment of the present invention, the compound is selected from the group consisting of 2-fluoro-phenylaminoethane-hydrazide, 4-fluoro-phenylaminoethane-hydrazide, 2,4-di-fluoro-phenylaminoethane-hydrazide, 4-chloro-phenylaminoethane-hydrazide, 3-chloro-4-fluoro-phenylaminoethane-hydrazide, 3-bromo-4-fluoro-phenylaminoethane-hydrazide, 4-methyl-phenylaminoethane-hydrazide, phenylaminoethane-hydrazide, 2-[(4-chlorophenyl)sulfanyl]acetohydrazide, 2-(4-fluorophenoxy)acetohydrazide, 2-(2-bromophenoxy)acetohydrazide, N-(2-fluorophenyl)glycin, 2-[(4-chlorophenyl)amino]acetic acid and 3-(2-hydroxyphenyl)propanohydrazide.

With the compounds according to the invention, in particular inflammatory diseases can be treated, the cause of which can be found in excessive EPO activity. Eosinophilic granulocytes and EPO are components of the unspecific immune defense. Particularly in case of inflammatory processes, there are accumulations of these white blood cells, which can also cause chronic inflammations. The inflammatory disease preferably is selected from the group consisting of bronchial asthma, multiple sclerosis, cystic fibrosis, ulcerative colitis, Crohn's disease, rhinitis, endometriosis, sinusitis, eosinophilic esophagitis, Shulman's syndrome (eosinophilic fasciitis), endocarditis, Churg-Strauss syndrome, dermatoses, preferably herpes gestationis or eosinophilic dermatosis, Hand-Schüller-Christian disease (ASCD), cardiovascular diseases, preferably endocarditis and hypertension due to inflammatory processes of the vascular walls.

Overview over exemplary diseases caused by eosinophil peroxidase (EPO), or in the course of which EPO is involved, respectively:

| Disease | Entity | Literature |
|---|---|---|
| Bronchial asthma | Chronic inflammatory disease of | (1), (4), (7), |
| | the airways, allergy | (15) |
| Eosinophilic der- | Different dermatological clinical | (2) |
| matosis | pictures | |
| Endometriosis | Hormone-related cramp-like pain | (3) |
| | by cervical mucus “islands” | |
| Ulcerative colitis | Chronic enteritis | (1), (5), (6) |
| Crohn's disease | Chronic enteritis | (5) |
| Sinusitis | Chronic inflammation of the sinuses/ | (9), (10) |
| | nasal catarrh, cold | |
| Rhinitis | Nasal catarrh, cold | (15), (16) |
| Cystic fibrosis | Genetically caused respiratory | (1) |
| | disease | |
| Eosinophilic | Chronic inflammation of the | (11) |
| esophagitis | esophagus | |
| Shulman's syn- | Chronic inflammation of the | (12) |
| drome—eosino- | connective tissue, edemas, muscle | |
| philic fasciitis | weakness, pain | |
| Endocarditis | Inflammation of the heart's inner | (13) |
| | membrane: weakness, fever | |
| Churg-Strauss | Inflammation of the small blood | (14) |
| syndrome | vessels: clinical picture of | |
| | rhinitis/asthma | |

(1) Davies M J, et al. Antioxidants & Redox Signaling. 10, 2008: 1199-1234.
(2) Wozel G. Hautarzt 58, 2007: 347-359.
(3) Blumenthal R D. et al., Exp. Rev. Mol. Med. 3, 2001: 1-12.
(4) Mitra S N, et al. Redox Rep. 5, 2000: 215-224.
(5) Wang J, et al. Arch Biochem Biophys 445, 2006: 256-260.
(6) Forbes E, et al. J Immunology 172, 2004: 5664-5675.
(7) Heinecke J W. J Clin Invest. 105, 2000: 1331-1332.
(8) Corry D B, et al. Immunol Res. 33, 2005: 35-52.
(9) Bernardes J F, et al. Otolaryngol Head Neck Surg. 131, 2004: 69-703.
(10) Bachert C, et al. Acta Otorhinolaryngol Belg. 51, 1997: 209-217.
(11) Straumann A, et al. Schweiz Med Forum 8, 2008: 724-728.
(12) Akanay-Diesel S, et al. Der Hautarzt 60, 2009: 278-281.
(13) Slungaard A, et al. J Exp Med. 173, 1991: 117-125.
(14) Eustace J A, et al. J Am Soc Nephrol 10, 1999: 2048-2055.
(15) Janeway's Immunobiology, ISBN 0-8153-4123-7, Garland Science, Taylor & Francis Group, 2008, 7th Edition: 566-583.
(16) Nielsen L P, et al. Allergy 64, 2009: 733-337.

In various inflamed organs and tissues as well as secretions obtained therefrom, EPO and/or its reaction products (e.g. nitrated, brominated lipids, proteins, DNA) could be detected. This, on the one hand, verifies the passive immune response by EPO within the scope of phagocytosis, on the other hand, it also massively shows the tissue-destroying effect of EPO and its reaction products. For example, in the sputum of asthma patients, EPO could be detected radio-immunologically, as well as 3-bromotyrosine by means of gas chromatography mass spectroscopy (GC-MS) (Aldridge et al. Free Radical Biology & Medicine 33 (2002) 847-856).

In an animal model (rat), it was demonstrated that, in the presence of bromide, EPO is a cause of endocarditis (Slungaard, A. et al. J Exp Med. 173 (1991) 117-26). Endocarditis is an inflammation of the heart's inner membrane lining the heart cavities and the portion of the arteries and veins close to the heart and also forming the structure of the heart valve leaflets. In principle, each human being can come down with endocarditis, and untreated, the course of the disease is mostly fatal. Antibiotics can be used for treatment of endocarditis.

Furthermore, ulcerative colitis is a disease caused by EPO. Wang et al. observed that EPO-free mice (EPO knock-out mouse line) compared to the wildtype hardly come down with ulcerative colitis. Crohn's disease, too, is a chronic inflammatory disease of the intestinal area, which is associated with the unspecific immune defense and EPO (Wang, J. et al. Arch Biochem Biophys 445 (2006) 256-260).

In allergic diseases like rhinitis (inflammation of the nasal mucosa), too, EPO is decisively involved (Hrdlickova, B. et al. Int Arch Allergy Immunol. 150 (2009) 184-91).

Furthermore, EPO is involved in the development of skin diseases (dermatoses), like herpes gestationis, a blistering autoimmune disease developing within the scope of pregnancy. Eosinophilic dermatoses frequently also occur in other mammals (dogs, cats) (Scheman, A J. et al. Arch Dermatol. 125 (1989) 1079-83).

Hodgkin's lymphoma (synonym: Hodgkin's disease or lympho-granulomatosis, abbreviated HD) is a malignant tumor of the lymphatic system. In examinations with radioactively labeled monoclonal antibodies against EPO directly at the site of the tumor, it showed that EPO is involved in apoptosis (Samoszuk, M K. et al. J Nucl Med. 34 (1993) 1246-53).

The Hand-Schüller-Christian disease (HSCD) mostly affects 2- to 5-year old children, adolescents and middle-aged adults. This form constitutes about 15-40% of langerhans-cell-histiocytoses. In about 30% of the people affected, there is systemic infestation affecting liver, spleen, lungs, skin and lymph nodes. The classic Hand-Schüller-Christian triad with bone lesions, exophthalmos and diabetes insipidus occurs rather rarely. With systemic infestation of multiple organs, there is a bad prognosis and the necessity of an aggressive chemotherapy and possibly stem cell transplantation. Otherwise, the disease can recede on its own, if necessary with chemotherapy. In studies, a massive release of EPO was determined. Ultimately, EPO is the cause for the massive tissue damaging caused within the scope of this disease (Zabucchi, G. et al. J Pathol. 163 (1991) 225-31).

The compounds according to the invention can be administered in a different manner. Depending on the disease, the compounds can be administered systemically or locally. The compounds according to the invention, in particular phenylaminoethane-hydrazide (PAEH) or its derivatives, respectively, therefore preferably are formulated in an intravenous, intracavitary, oral, intraperitoneal, inhalation and topical dosage form.

According to the type of administration, the compound according to the invention, in particular phenylaminoethane-hydrazide or its derivatives, respectively, is preferably present in the form of an infusion, tablet, capsule, cream, gel, emulsion or patch.

Depending on the dosage form, the pharmaceutical composition according to the invention comprises, beside the compounds according to the invention, excipients, like, e.g., disintegrating agents and stabilizers, carriers and diluents.

Examples for common excipients, carriers and diluents are gelatine, natural sugars (like sucrose or lactose, lecithin, pectin, starch (e.g. corn starch) as well as starch derivatives, cyclodextrins and cyclodextrin derivatives, polyvinylpyrrolidone, gelatine, gum arabic, alginic acid, tylose, talcum, lycopodium, silicic acid (e.g. colloidal), fructose, tragacanth, sodium chloride, stearates, magnesium and calcium salts of fatty acids with 12 to 22 C-atoms, in particular of the saturated ones (e.g. stearates), polyethylene glycol with a mean molecular weight between 200 and 20,000, preferably between 200 and 5,000, in particular between 200 and 1,000, or their mixtures, and/or polymerisates of vinylpyrrolidone and/or mixed polymerisates of vinylpyrrolidone and vinylacetate. Esters of aliphatic saturated or unsaturated fatty acids (2 to 22 C-atoms, in particular 10 to 18 C-atoms) with monovalent aliphatic alcohols (1 to 20 C-atoms) or multivalent alcohols like glycols, glycerol, diethyleneglycol, pentaerythrite, sorbitol, mannitol, etc., which may also be etherified, if necessary, benzylbenzoate, dioxolanes, glycerol formals, tetrahydrofurfurylalcohol, polyglykolether with C₁ to C₁₂ alcohols, dimethylacetamide, lactamides, lactates, ethylcarbonates, silicones (in particular medium-viscous polydimethylsiloxanes), calcium carbonate, sodium carbonate, calcium phosphate, sodium phosphate, magnesium carbonate, gum arabic, alginic acid, stearates, fats and substances with a similar effect. For solutions, like e.g. infusions, various buffer systems can be used.

A further aspect of the present invention relates to a pharmaceutical composition comprising a compound like described herein for treatment and/or prevention of diseases, in particular inflammatory diseases, which are related to eosinophil peroxidase.

The pharmaceutical composition according to the invention is preferably present in the form of an infusion, tablet, capsule, cream, gel, emulsion or patch.

A still further aspect of the present invention relates to the use of the compounds according to the present invention for the manufacture of medication for treatment and/or prevention of diseases, in particular inflammatory diseases, which are related to eosinophil peroxidase.

A further aspect of the present invention relates to a method for the treatment and/or prevention of diseases, in particular inflammatory diseases, which are related to eosinophil peroxidase, by administration of one or several of the compounds according to the invention.

The present invention is explained in more detail on the basis of the following examples, however, without being restricted to these.

### EXAMPLES

### Example 1

In order to test to what extent the substances according to the invention are able to inhibit EPO, the substances were tested for their inhibitory potential. In that, the IC₅₀ value was determined as a comparable parameter. In that, IC₅₀ is that inhibitor concentration, which is required to inhibit an enzyme, here EPO, by 50%. This concentration is determined UV/Vis spectrophotometrically at 290 nm in the steady-state with a monochlorodimedon (MCD) assay.

Determination of the Inhibitory Effect

IC₅₀ Value Determination

Eosinophil peroxidase forms a multiplicity of different enzyme intermediates and is able to catalyze a high number of redox reactions. The physiological role of EPO is the oxidation of bromide or thiocyanate, respectively, to hypobromous acid or hypothiocyanate, respectively (also called halogenation cycle). And it is exactly this reaction that has to be inhibited. In the presence of phenolic substances, however, the enzyme can also undergo the so-called peroxidase cycle.

In order to determine the properties of the substances according to the invention to be inhibited, a method was used, in which the bromination activity is examined.

Bromination Activity

The extent of inhibition of the physiological bromide oxidation was photometrically determined using monochlorodimedon. The halogenation rate (initial inclination of the curve at 290 nm) with inhibitor was related to a blind value (without inhibitor), and therefrom the inactivation rate (in %) was determined. This was entered into a diagram (y-axis) opposite the inhibitor concentration (x-axis), and from the hyperbolic fit of the curve, the IC₅₀ value for each inhibitor was determined.

100 mM of phosphate buffer, pH 7.0

100 μM of monochlorodimedon

100 mM of bromide

20 nM of EPO

100 μM of HOOH

0.001-500 μM of inhibitor

Phenylaminoethane-Hydrazides

In the examination of various substance groups, which due to their structure presumably fit into the catalytic center of EPO (and the homologous LPO), and there also inhibit the activity, it turned out that the substance group of the phenylaminoethane-hydrazides (III), but in particular of their derivatives and halogenated derivatives thereof, are very good selective inhibitors of EPO. Examples for respective derivatives, but above all halogenated derivatives, have to be stated as follows: on the basis of several examples, Table 2 shows the selectivity of the phenylaminoethane-hydrazides for EPO (and also for the homologous LPO), but not for MPO:

TABLE 2
Example for phenylaminoethane-hydrazide derivatives and
the inhibitory potential (IC₅₀: concentration at which 50% of
the enzyme activity are inhibited)

| Phenylaminoethane-hydrazides | | EPO + Br | LPO + Br | MPO + Br | MPO + Cl |
|---|---|---|---|---|---|
| (PAEHs) | Structural formula | [μM] | [μM] | [μM] | [μM] |
| (1) 4-fluoro- phenylaminoethane-hydrazide | 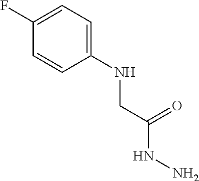 [SMILES: NNC(=O)CNc1ccc(F)cc1] | 0.240 | 0.540 | 4.120 | 5.430 |
| (2) 4-chloro- phenylaminoethane-hydrazide | 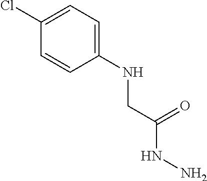 [SMILES: NNC(=O)CNc1ccc(Cl)cc1] | 0.024 | 0.030 | 1.200 | 1.970 |
| (3) 2-fluoro- phenylaminoethane-hydrazide | 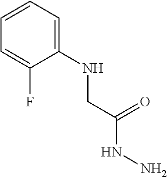 [SMILES: NNC(=O)CNc1ccccc1F] | 0.009 | 0.100 | 1.900 | 8.800 |
| (4) 4-fluoro- 3-chloro- phenylaminoethane-hydrazide | 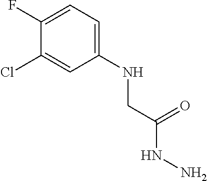 [SMILES: NNC(=O)CNc1ccc(F)c(Cl)c1] | 0.019 | 0.140 | 0.547 | 2.400 |
| (5) 3-bromo- phenylaminoethane-hydrazide | 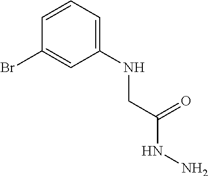 [SMILES: NNC(=O)CNc1cccc(Br)c1] | 0.017 | 0.040 | 1.600 | 3.700 |
| (6) Non-halogenated phenylaminoethane-hydrazide | 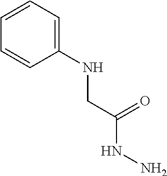 [SMILES: NNC(=O)CNc1ccccc1] | 2.290 | 4.967 | 84.56 | 46.04 |
| (7) 2,4-di-fluoro- phenylaminoethane-hydrazide | 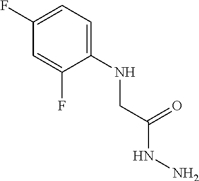 [SMILES: NNC(=O)CNc1ccc(F)cc1F] | 0.034 | 0.322 | 2.040 | 6.550 |
| (8) Non-halogenated 4-methyl- phenylaminoethane-hydrazide | 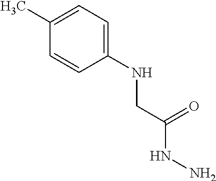 | 2.270 | 2.773 | 29.40 | 32.19 |

The compound (3) 2-fluorophenyl-NH-ethanehydrazide has an IC₅₀ value for EPO of 0.009 μM, but for MPO a substantially higher IC₅₀ value of 1.900 or 8.800 μM, respectively. I.e., this substance represents a very good inhibitor for EPO, but not for MPO of the same enzyme family of human peroxidases.

Furthermore, it can be retrieved from Table 2 that halogenated phenylaminoethane-hydrazide derivatives have a stronger inhibitory effect than non-halogenated ones.

Compound (6) phenylaminoethane-hydrazide shows an IC₅₀ value of 2.290 μM. This potential can already result in therapeutic application as inhibitor, with good tolerability. However, example number (3) 2-fluorophenyl-NH-ethanehydrazide shows more than the 200-fold potential with an IC_{H} value of 0.009 μM. Thereby, very low therapeutic concentrations are possible, which thereby also minimize possibly occurring undesired side effects.

### Example 2

In a further test series, it was examined to what extent further substances of the general formula (I) are able to inhibit the activity of EPO. As an example, isoniazide (pyridine-4-carbohydrazide) was used, in which Rx in the general formula (I) represents a pyridine residue. The tests were performed as represented in Example 1.

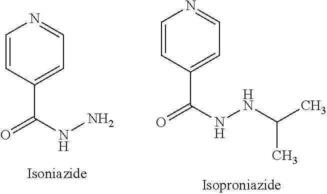

It was determined that isoniazide has an IC₅₀ value of 6.04 μM.

In order to examine the influence of the free amino group at the hydrazide residue of the general formula (I) on the inhibitory properties of the substances according to the invention on EPO, a derivative of isoniazide, namely N′-isopropylisonicotinohydrazide (iproniazide), was examined. In that, it was surprisingly determined that iproniazide has an IC₅₀ value of more than 500 μM.

This verifies that for the strong inhibition of the EPO activity, beside other properties (II), the free amino group of the substances according to the general formula (I) is decisive in any case. This could be impressively demonstrated at the example of the structurally related substances isoniazide and iproniazide. Derivatization of the free amino groups results in a loss of the inhibitory strength.

### Example 3

In a further test series, which was performed according to the same protocol as stated in Example 1, further compounds according to the invention were examined for their abilities to inhibit eosinophil peroxidase. The results of these tests and the compounds used therein can be retrieved from the following table.

| # | Name | MG | CAS | IC50 EPO/Br (μM) |
|---|---|---|---|---|
| PD01 | N-(2-fluorophenyl)glycin 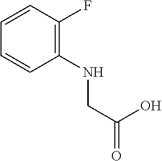 [SMILES: O=C(O)CNc1ccccc1F] | 169.155423 | 5319-42-6 | 1.0 |
| PD02 | 2-[(4-chlorophenyl)amino]acetic acid 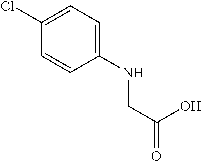 [SMILES: O=C(O)CNc1ccc(Cl)cc1] | 185.61146 | 5465-90-7 | 0.2 |
| PD06 | 2-[(4-chlorophenyl)sulfanyl]acetohydrazide 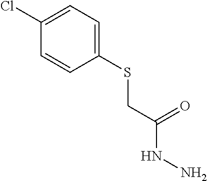 [SMILES: NNC(=O)CSc1ccc(Cl)cc1] | 216.69073 | 75150-40-2 | 1.2 |
| PD09 | 2-(4-fluorophenoxy)acetohydrazide 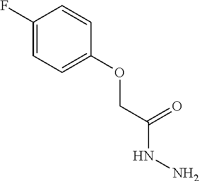 [SMILES: NNC(=O)COc1ccc(F)cc1] | 184.17153 | 1737-62-8 | 2.7 |
| PD17 | 2-(2-bromophenoxy)acetohydrazide 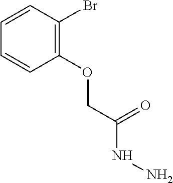 [SMILES: NNC(=O)COc1ccccc1Br] | 245.07566 | 328085-17-2 | 3.9 |
| PD20 | 3-(2-hydroxyphenyl)propanohydrazide 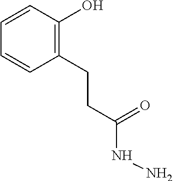 [SMILES: NNC(=O)CCc1ccccc1O] | 180.20648 | 24535-13-5 | 3.1 |

### Example 4

In order to show the pharmacological effect of the compounds according to the invention, animal models can be used. Using animal models, it is possible to verify by way of experiments, to what extent pharmacologically active agents have respective effects.

1. Bronchial Asthma

Several factors are responsible for manifestation and progress of bronchial asthma (1): allergens, emotional stress, physical exertion, cold air and all combinations of these factors. The pathophysiological response is very complex, but there is a “red thread” to our target, EPO. T-helper 2 (Th2) cells result in interleukin release, in particular IL-5, which causes the release of eotaxins. These result in the migration of eosinophilic granulocytes to the lung site of action. The increased IgE levels and IgE receptors at the eosinophils with the allergy result in degranulation and release of proteins with a 60% portion of EPO. EPO catalyzes the oxidation of halides and thiocyanate, wherein highly reactive oxidation products are formed, which are released for the defense against parasites and microorganisms, but (in case of asthma and other chronic diseases) also have a tissue-destructing effect.

Therefore, a “chronic model” is required, wherein it must be verified, whether this mechanism also takes place and is approximated to the human courses. With this model, the effect of EPO inhibitors can then be tested.

For verification of the effect, respective animal models are used, which, however, especially in connection with asthma and EPO, are complex.

Approach:

Balb/c mice with a body weight of 18-21 g are kept in an acclimatization phase of one week.

The irrelevance of ovalbumin and induction of asthma (allergic inflammation of the airways) is known, therefore stimulation takes place with house dust mites or grass pollen. Over 7 weeks, the allergen is applied transnasally every day. This stimulation directly results in asthma symptoms with AHR (acute airway hyper-responsiveness) and eosinophilic inflammation of the airways (Johnson et al. 2004, Am J Respir Crit. Care Med 169:378-385; Johnson et al. 2008, Am J Physiol Lung Cell Mol Physiol 295:L780-L788).

Using ELISA, inflammation parameters, eosinophilic granulocytes and EPO are finally measured in the BALB (bronchioalveolar liquid) supernatant. Where EPO is active, these individuals are divided into therapy and control groups. The therapy group received the compounds according to the invention (1-10 mg/kg KG daily), while the control group receives a placebo. As parameters for the development of the allergy and chronic inflammation of the airways and lungs, among other things, the number of exacerbations (severe attack) and the extent of the AHR are used. A third group can be treated with dexamethason (among others) in a conventional manner.

2. Rhinitis and Sinusitis

The effects of the compounds according to the invention with diseases of the sinuses and ethmoid bones can be determined with the same animal model like bronchial asthma.

3. Endometriosis

Animal models for the effectiveness test of drug candidates for endometriosis are well established and easy to perform. Rat (Neto J N, Coelho T M, Aguiar G C, Carvalho L R, de Araújo A G, Girão M J, Schor E. Experimental endometriosis reduction in rats treated with Uncaria tomentosa (cat's claw) extract. Eur J Obstet Gynecol Reprod Biol. 2010 Oct. 26.) and mouse (Lu Y, et al. Hum Reprod. 25 (2010):1014-25) are the common test animals. In that, human fragments of endometriosis tissue are transplanted into the test animals. After an adaptation period of three to four weeks, the compounds according to the invention can be “simply” tested and compared with a placebo group or with a group treated with a conventional therapy, respectively.

4. Endocarditis

Is an infectious disease of the heart's inner membrane and can be well simulated in the rat model (Singh K V, et al. PLoS Pathog. 2010 Jan. 8; 6(1):e1000716).

5. Chronic Inflammatory Intestinal Diseases (Inflammatory Bowel Diseases, e.g.: Crohn's Disease and Ulcerative Colitis)

In that, colon cells are taken from mice and prepared for further examinations (Weigmann B, et al. Nat Protoc 2 (2007):2307-11.). In that, the peroxidase activity can be tested using an enzymatic MCD (monochlorodimedon) assay, or following electrophoretic separation as active staining in the gel.

6. Cystic Fibrosis

Easily performable test with mice. Since cystic fibrosis is also associated with infection, the test animals are infected and treated following outbreak of the disease (drug candidate placebo—conventional) (Wang Y, et al. Respir Res. 2010 Nov. 30; 11:166; Guilbault C, et al. Lab Anim. 2005 July; 39(3):336-52).

1. A compound of the general formula (III): 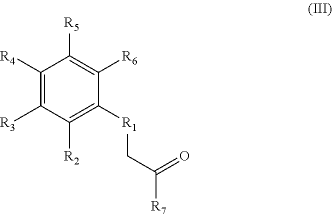
for use in the treatment and/or prevention of a disease related to eosinophil peroxidase, wherein
R₁ is CH₂, NH, O, S or a single bond,
R₂, R₃, R₄, R₅ and R₆ independently of one another are H, OH, F, Cl, Br, I or a C₁ to C₅ alkyl group, and
R₁ is H, OH, NH₂, NH—NH₂ or CH₃.

2. The compound according to claim 1, wherein R₁ is NH and R₇ is NH—NH₂ and said compound has the general formula (IV).

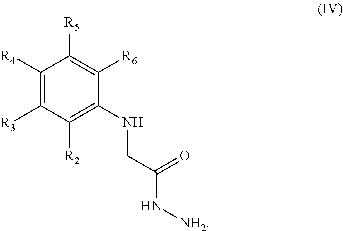

3. The compound according to claim 1, wherein said C₁ to C₅ alkyl group is selected from the group consisting of CH₃ and CH₂CH₃.

4. The compound according to claim 1, wherein R₁ is CH₂, NH, O or S, R₂ is F or H, R₃ is Cl, Br or H, R₄ is Cl, F, CH₃ or H, R₅ and R₆ are H, and R₇ is OH or NH—NH₂.

5. The compound according to claim 1, wherein said compound is selected from the group consisting of 2-fluoro-phenylaminoethane-hydrazide, 4-fluoro-phenylaminoethane-hydrazide, 2,4-di-fluoro-phenylaminoethane-hydrazide, 4-chloro-phenylaminoethane-hydrazide, 3-chloro-4-fluoro-phenylaminoethane-hydrazide, 3-bromo-4-fluoro-phenylaminoethane-hydrazide, 4-methyl-phenylaminoethane-hydrazide, phenylaminoethane-hydrazide, 2-[(4-chlorophenyl)sulfanyl]acetohydrazide, 2-(4-fluorophenoxy)acetohydrazide, 2-(2-bromophenoxy)acetohydrazide, N-(2-fluorophenyl)glycine, 2-[(4-chlorophenyl)amino]acetic acid, and 3-(2-hydroxyphenyl)propanohydrazide.

6. The compound according to claim 1, wherein the disease related to eosinophil peroxidase is selected from the group consisting of bronchial asthma, multiple sclerosis, cystic fibrosis, ulcerative colitis, Crohn's disease, rhinitis, endometriosis, sinusitis, eosinophilic esophagitis, Shulman's syndrome (eosinophilic fasciitis), endocarditis, Churg-Strauss syndrome, dermatoses, herpes gestationis, eosinophilic dermatosis, Hand-Schüller-Christian disease (ASCD), cardiovascular diseases, endocarditis and hypertension due to inflammatory processes of the vascular walls.

7. The compound according to claim 1, wherein said compound is provided in an intravenous, intracavitary, oral, intraperitoneal, inhalation or topical dosage form.

8. The compound according to claim 1, wherein said compound is provided in the form of an infusion, tablet, capsule, cream, gel, emulsion or patch.

9. The compound according to claim 1, wherein said compound is administered at an amount of 0.01 to 2,000 mg/kg of body weight.

10. A pharmaceutical composition comprising at least one compound according to claim 1, for the treatment of a disease related to eosinophil peroxidase.

11. The pharmaceutical composition according to claim 10, wherein said disease is selected from the group consisting of bronchial asthma, multiple sclerosis, cystic fibrosis, ulcerative colitis, Crohn's disease, rhinitis, endometriosis, sinusitis, eosinophilic esophagitis, Shulman's syndrome (eosinophilic fasciitis), endocarditis, Churg-Strauss syndrome, dermatoses, herpes gestationis, eosinophilic dermatosis, Hand-Schüller-Christian disease (ASCD), cardiovascular diseases, endocarditis and hypertension due to inflammatory processes of the vascular walls.

12. The pharmaceutical composition according to claim 10, wherein said compound is provided in an intravenous, intracavitary, oral, intraperitoneal, inhalation or topical dosage form.

13. The pharmaceutical composition according to claim 10, wherein said compound is provided in the form of an infusion, tablet, capsule, cream, gel, emulsion or patch.

14. Use of a compound according to claim 1 for the manufacture of medication for the treatment and/or prevention of a disease related to eosinophil peroxidase.

15. Use of a compound according to claim 1 for inhibition of eosinophil peroxidase.

16. The compound according to claim 2, wherein said C₁ to C₅ alkyl group is selected from the group consisting of CH₃ and CH₂CH₃.

17. The compound according to claim 2, wherein R₁ is CH₂, NH, O or S, R₂ is F or H, R₃ is Cl, Br or H, R₄ is Cl, F, CH₃ or H, R₅ and R₆ are H, and R₇ is OH or NH—NH₂.

18. The compound according to claim 3, wherein R₁ is CH₂, NH, O or S, R₂ is F or H, R₃ is Cl, Br or H, R₄ is Cl, F, CH₃ or H, R₅ and R₆ are H, and R₇ is OH or NH—NH₂.

19. The pharmaceutical composition according to claim 1, wherein said disease is an inflammatory disease.
